(19) United States
(12) Patent Application Publication
(10) Pub. No.: US 2026/0069246 A1
(43) Pub. Date: Mar. 12, 2026

(54) MULTI-PURPOSE ULTRASONIC TRANSDUCER

(71) Applicant: Qisda Corporation, Taoyuan City (TW)

(72) Inventor: Fu-Sheng JIANG, Taoyuan City (TW)

(73) Assignee: Qisda Corporation, Taoyuan City (TW)

(21) Appl. No.: 19/269,281

(22) Filed: Jul. 15, 2025

(30) Foreign Application Priority Data
Sep. 12, 2024 (TW) 113134624

(51) Int. Cl. A61B 8/00

(52) U.S. Cl. CPC A61B 8/4494; A61B 8/4444; A61B 8/4488

(57) ABSTRACT

An ultrasonic transducer is provided, which includes a first transducer and a second transducer. The first transducer includes several first array units arranged along a first direction to form a first array. The first direction is a direction of a long axis of the ultrasonic transducer. The second transducer includes several second array units arranged along the first direction to form a second array. The second array and the first array are interleaved and separated from each other. One of the second array units is sandwiched between adjacent two of the first array units. The first transducer and the second transducer have a common reference coordinate and have a function of a multi-focus lenses along the second direction. The second direction is a direction of a short axis of the ultrasonic transducer, which is substantially perpendicular to the first direction.

This application claims the benefit of Taiwan application Serial No. 113134624, filed Sep. 12, 2024, the disclosure of which is incorporated by reference herein in its entirety.

### TECHNICAL FIELD

The present disclosure relates to a detection device, particularly relates to an ultrasonic transducer for detecting images of tissues and organs of human or animals.

### BACKGROUND

With the development of medical technology, various forms of image detection devices have been widely used to detect images of tissues and organs of human or animals. Among various image detection devices actually used in medical institutions, ultrasonic transducers are often employed for ultrasonic detection, so as to obtain images of diseased organs or tissues of human body.

A conventional ultrasonic transducer may have functions of linear array(s) (LA) and phased array(s) (PA). Linear array(s) operate based on one set of reference coordinate or center-line, while phased array(s) operate based on another set of reference coordinate or center-line. Therefore, when switching between a linear array mode or a phased array mode, the reference coordinate or center-line must be re-aligned.

Alternatively, the ultrasonic transducer must be provided with two probes to respectively perform functions of linear array(s) and phased array(s), which will increase operational complexity and hardware cost of the ultrasonic transducer. Furthermore, when the conventional ultrasonic transducer is in operation, image quality of the linear array(s) may be affected by artifacts.

In view of the above technical problems, it is desirable to provide an improved ultrasonic transducer that can perform functions of linear array(s) and phased array(s) (or even the functions of a multi-focal lens) based on the same reference coordinate or center-line with a single probe, and can be adapted to different operating frequencies and different resolutions.

### SUMMARY

According to one embodiment of the present disclosure, an ultrasonic transducer is provided. The ultrasonic transducer includes a first transducer and a second transducer. The first transducer includes several first array units which are arranged along a first direction to form a first array. The first direction is a direction of a long axis of the ultrasonic transducer. The second transducer includes several second array units which are arranged along the first direction to form a second array. The second array and the first array are disposed in an interleaving manner and separated from each other. One of the second array units is sandwiched between corresponding adjacent two of the first array units. The first transducer and the second transducer have a common reference coordinate, and the first transducer and the second transducer have a function of a multi-focal lens along a second direction, the second direction is a direction of a short axis of the ultrasonic transducer and is substantially perpendicular to the first direction.

According to another embodiment of the present disclosure, an ultrasonic probe is provided. The ultrasonic probe includes a gripping handle and an ultrasonic transducer. The ultrasonic transducer is disposed at one end of the gripping handle.

### BRIEF DESCRIPTION OF THE DRAWINGS

FIG. 1A is a top view of an ultrasonic probe 100 according to an embodiment of the present disclosure.

FIG. 1B is a front view of the ultrasonic probe 100 of FIG. 1A.

FIG. 2A is a top view of the two sets of transducers 101 and 201 included in the ultrasonic transducer 1000 of FIG. 1B.

FIG. 2B is a schematic diagram showing a projection of one of the array units L1-L64 and one of the array units R1-R64 in FIG. 2A.

FIG. 3A is a top view of the transducer 102 and the transducer 202 according to another embodiment of the present disclosure.

FIG. 3B is a schematic diagram showing a projection of one of the array units L1-L64 and one of the array units R1-R64 in FIG. 3A.

FIG. 4A is a top view of the transducer 103 and the transducer 203 according to another embodiment of the present disclosure.

FIG. 4B is a schematic diagram showing projections of one of the array units L1-L64 and one of the array units R1-R64 in FIG. 4A.

FIG. 5A is a schematic diagram showing the transducers 101 and 201 in FIG. 2A simulate the functions of linear array(s) and phased array(s).

FIG. 5B is a schematic diagram showing the transducer 101 and the transducer 201 in FIG. 2A simulate the function of phased array(s).

FIG. 6 is a schematic diagram showing the operations of the transducer 104 and the transducer 204 according to yet another embodiment of the present disclosure.

FIG. 7 is a schematic diagram showing the transducer 104 and the transducer 204 in FIG. 6 are simulated as a multi-focal lens len1.

FIG. 8 is a schematic diagram showing another embodiment in which the ultrasonic transducer 1000 is simulated as the multi-focal lens len1.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

The ultrasonic transducer 1000 of the present disclosure is used to generate an ultrasonic signal to detect a target object. The ultrasonic signal reflected from the target object can be converted into electrical energy to generate an electrical signal, and the ultrasonic transducer 1000 generates a two-dimensional image of the target object according to the electrical signal. The target object is, for example, a tissue or an organ of different parts of the human body, which may include a heart, a carotid artery, a stomach, and an intestine, etc. The ultrasonic transducer 1000 of the present disclosure integrates multiple functions, including at least the function of a phased array (PA) and the function of a linear array (LA), and can simulate the function of a convex linear array (CLA). Furthermore, the intersection or union of the phased array and the linear array can further simulate the function of a multi-focal lens. The multiple functions of the ultrasonic transducer 1000 can be adapted to different operating frequencies, different resolutions, and different focal lengths for imaging, so as to facilitate the detection of tissues or organs in different locations, and can be applied to various purposes.

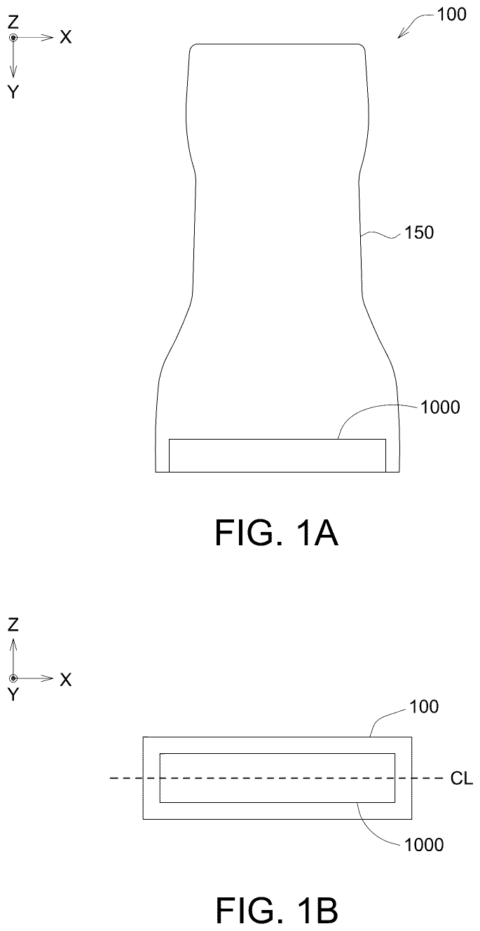

FIG. 1A is a top view of an ultrasonic probe 100 according to an embodiment of the present disclosure, and FIG. 1B is a front view of the ultrasonic probe 100 of FIG. 1A. Please refer to both FIGS. 1A and 1B, one end of the ultrasonic probe 100 includes an ultrasonic transducer 1000, for example, the ultrasonic probe 100 includes a gripping handle 150, and the ultrasonic transducer 1000 is disposed at one end of the gripping handle 150. The ultrasonic transducer 1000 may operate based on a reference coordinate formed by an X-axis, a Y-axis, and a Z-axis, in which the X-axis is also referred to as a “long axis X” and the Z-axis is also referred to as a “short axis Z”. The ultrasonic transducer 1000 extends substantially along the long axis X, and has a center-line CL (the center-line CL is parallel to the long axis X). The ultrasonic probe 100 can transmit ultrasonic signals to a target object through the ultrasonic transducer 1000, and receive ultrasonic signals reflected by the target object. When the phased array, the linear array and the multi-focal lens of the ultrasonic transducer 1000 are in operation, the phased array operates based on the direction of the long axis X, and the linear array operates based on the direction of the short axis Z, for example.

More specifically, the ultrasonic transducer 1000 includes a plurality of transducers, such as array units made of piezoelectric materials, which are employed for transmitting, receiving and converting the ultrasonic signals. In this embodiment, the ultrasonic transducer 1000 may include two sets of transducers, which may facilitate the simulation and execution of the functions of the phased array, the linear array, and the multi-focal lens. Configuration and operation of two sets of transducers will be described in detail in the following paragraphs.

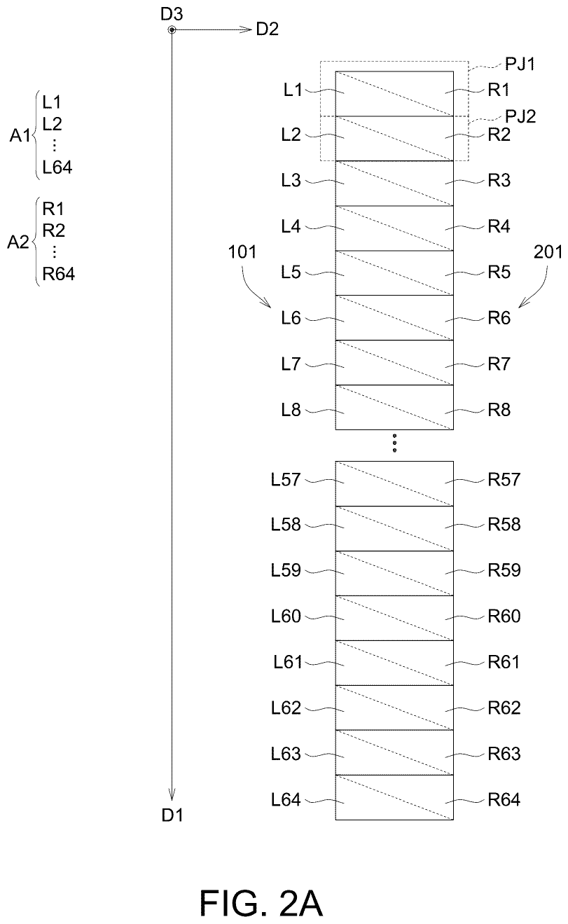

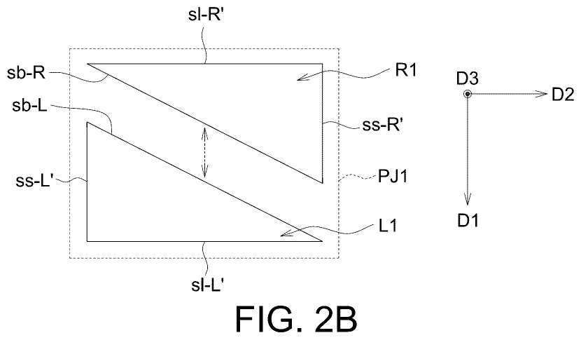

FIG. 2A is a top view of the two sets of transducers 101 and 201 included in the ultrasonic transducer 1000 of FIG. 1B. The transducer 101 is referred to as a “first transducer”, and the transducer 201 is referred to as a “second transducer”. The transducer 101 and the transducer 201 have a common reference coordinate, i.e., the reference coordinate formed by the long axis X, the short axis Z and the axis Y of FIG. 1B. In FIG. 2A, a direction D1 indicates the direction of the long axis X, a direction D2 indicates the direction of the short axis Z, and a direction D3 indicates the direction of the axis Y. The direction D1 is referred to as a “first direction”, and the direction D2 is referred to as a “second direction”. In this embodiment, the directions D1, D2 and D3 are substantially perpendicular to one another. In another embodiment, the transducer 101 and the transducer 201 may also be arranged in an arc shape, and hence the direction D2 does not need to be substantially perpendicular to the direction D1.

The transducer 101 includes a plurality of array units, for example, 64 array units L1-L64. Each of the array units L1-L64 is referred to as a “first array unit”. The array units L1-L64 are arranged along the direction D1 to form an array A1, which may be referred to as a “first array”. Similarly, the transducer 201 also includes a plurality of array units, for example, 64 array units R1-R64. Each of the array units R1-R64 is referred to as a “second array unit”. The array units R1-R64 are also arranged along the direction D1 to form an array A2, which may be referred to as a “second array”.

The array A1 formed by the array units L1-L64 and the array A2 formed by the array units R1-R64 are arranged in an interleaved manner. In the embodiment of FIG. 2A, one of the array units L1-L64 of the array A1 is sandwiched between adjacent two of the corresponding array units R1-R64 of the array A2. For example, the array unit L1 of the array A1 is sandwiched between two corresponding adjacent array units R1 and R2 of the array A2. A next array unit L2 of the array A1 is sandwiched between two corresponding adjacent array units R2 and R3 of the array A2.

Each of the array units L1-L64 of the array A1 and each of the array units R1-R64 of the array A2 are separated from each other. For example, the array unit L1 of the array A1 is separated from the corresponding array unit R1 and array unit R2 of the array A2, and the array unit L2 of the array A1 is separated from the corresponding array unit R2 and array unit R3 of the array A2. The dotted lines shown in FIG. 2A may represent gaps separating the array units L1-L64 from the array units R1-R64. The above-mentioned gaps may be formed during a manufacturing process of the piezoelectric materials of the transducer 101 and the transducer 201, for example, formed by semiconductor etching or laser cutting. An equivalent pitch between adjacent two of the array units L1-L64 is equal to an equivalent pitch between adjacent two of the array units R1-R64, which is a pitch P1. The pitch P1 is referred to as a “first pitch”. For example, the equivalent pitch between two adjacent array units L1 and L2 is pitch P1, and the equivalent pitch between two adjacent array units R1 and R2 is also pitch P1.

The operations of transducer 101 and transducer 201 along direction D2 can simulate the function of the multi-focal lens. Furthermore, the operations of the transducer 101 and the transducer 201 along the direction D2 and/or the direction D1 may simulate the functions of the linear array and/or the phased array. A first equivalent pitch of the linear array with reference to the direction D1 is equal to twice the pitch P1. Furthermore, a second equivalent pitch of the phased array with reference to the direction D1 is equal to the pitch P1.

Each of the array units L1-L64 and the array units R1-R64 has a projection with a specific shape along the direction D3, such as a triangular projection in FIG. 2A, or a step-shaped projection or fork-shaped projection in other embodiments.

FIG. 2B is a schematic diagram showing a projection of one of the array units L1-L64 and one of the array units R1-R64 in FIG. 2A (the array unit L1 and the array unit R1 are taken as examples for FIG. 2B). Please refer to both FIGS. 2A and 2B, the shape and area of the triangular projection of the array unit R1 may be equal to the triangular projection of the array unit L1. The triangular projection of the array unit R1 may be flipped along the direction D1 and then flipped along the direction D2 to obtain the triangular projection of the array unit L1.

The triangular projection of the array unit R1 has a short side ss-R′ along the direction D1, a long side sl-R′ along the direction D2, and a hypotenuse sb-R connected to the short side ss-R′ and the long side sl-R′. Similarly, the triangular projection of the array unit L1 has a short side ss-L′ along the direction D1, a long side sl-L′ along the direction D2, and a hypotenuse sb-L connected to the short side ss-L′ and the long side sl-L′.

The hypotenuse sb-R of the triangular projection of array unit R1 is disposed corresponding to (e.g., adjacent to) the hypotenuse sb-L of the triangular projection of array unit L1. The hypotenuse sb-R and the hypotenuse sb-L are separated from each other. The gap between the hypotenuse sb-R and the hypotenuse sb-L shown in FIG. 2B is the gap (indicated by a dotted line) separating the array unit L1 and the array unit R1 shown in FIG. 2A. Furthermore, the triangular projection of the array unit L1 and the corresponding triangular projection of the array unit R1 form a long strip projection PJ1, the triangular projection of the array unit L2 and the corresponding triangular projection of the array unit R2 form a long strip projection PJ2, and the like.

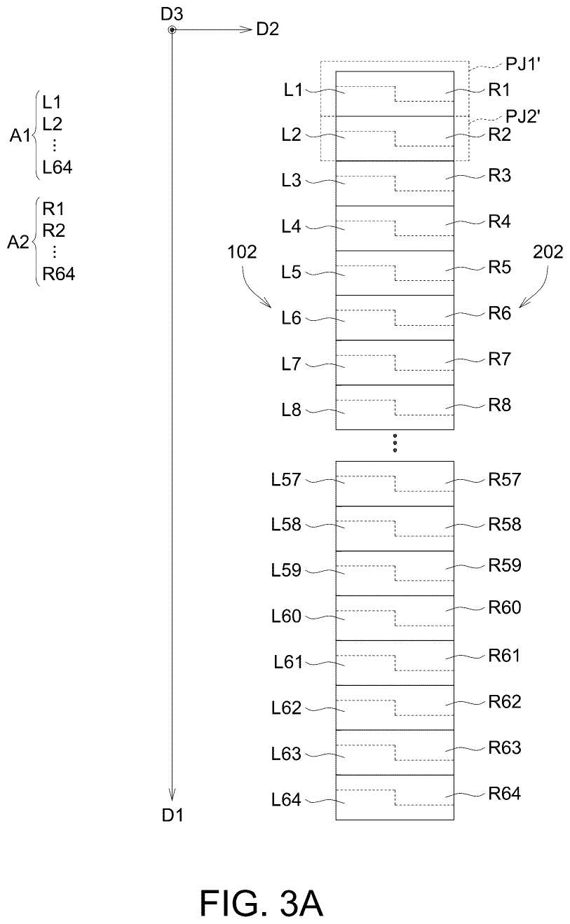

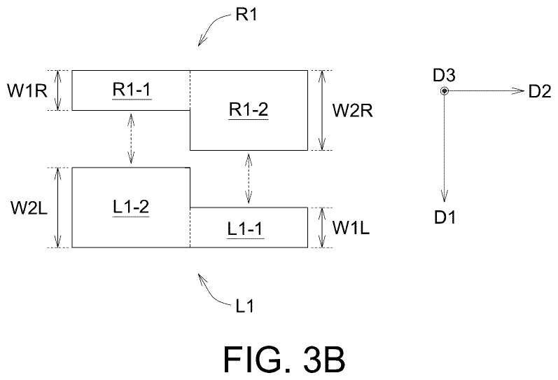

FIG. 3A is a top view of the transducer 102 and the transducer 202 according to another embodiment of the present disclosure. FIG. 3B is a schematic diagram showing a projection of one of the array units L1-L64 and one of the array units R1-R64 in FIG. 3A (the array unit L1 and the array unit R1 are taken as examples for FIG. 3B). Please refer to both FIGS. 3A and 3B, each of the array units L1-L64 and the array units R1-R64 has a step-shaped projection along the direction D3. Furthermore, the shape and area of the step-shaped projection of each of the array units L1-L64 may be equal to the step-shaped projection of each of the array units R1-R64. For example, the step-shaped projection of the array unit R1 may be flipped along the direction D1 and then flipped along the direction D2 to obtain the step-shaped projection of the array unit L1.

According to the step-shaped projections, the array A1 of the transducer 102 and the array A2 of the transducer 202 are arranged in an interleaved manner (similar to a manner of shaking-hands). Furthermore, one of the array units L1-L64 and a corresponding one of the array units R1-R64 may form a long strip projection. For example, the step-shaped projection of array unit L1 and the corresponding step-shaped projection of array unit R1 may form a long strip projection PJ1′, the step-shaped projection of array unit L2 and the corresponding step-shaped projection of array unit R2 may form a long strip projection PJ2′, and so on.

More specifically, the step-shaped projection of the array unit R1 includes a first portion R1-1 and a second portion R1-2. The first portion R1-1 extends along the direction D2 and has a width W1R along the direction D1. On the other hand, the second portion R1-2 also extends along the direction D2 and has a width W2R along the direction D1. The width W2R of the second portion R1-2 is greater than the width W1R of the first portion R1-1.

Similarly, the step-shaped projection of the array unit L1 corresponding to the array unit R1 also includes a first portion 1-1I and a second portion L1-2. The first portion L1-1 extends along the direction D2 and has a width W1L along the direction D1. On the other hand, the second portion L1-2 also extends along the direction D2 and has a width W2L along the direction D1. The width W2L of the second portion L1-2 is greater than the width W1L of the first portion L1-1.

The first portion R1-1 and the second portion R1-2 with the step-shaped projection of the array unit R1 are respectively arranged corresponding to the second portion L1-2 and the first portion L1-1 with the step-shaped projection of the corresponding array unit L1. For example, the first portion R1-1 with the step-shaped projection of the array unit R1 is adjacent to the second portion L1-2 with the step-shaped projection of the array unit L1. Furthermore, the second portion R1-2 with the step-shaped projection of the array unit R1 is adjacent to the first portion 1-1I with the step-shaped projection of the array unit L1. The step-shaped projection of the array unit L1 and the step-shaped projection of the array unit R1 are separated from each other and form a long strip projection PJ1′.

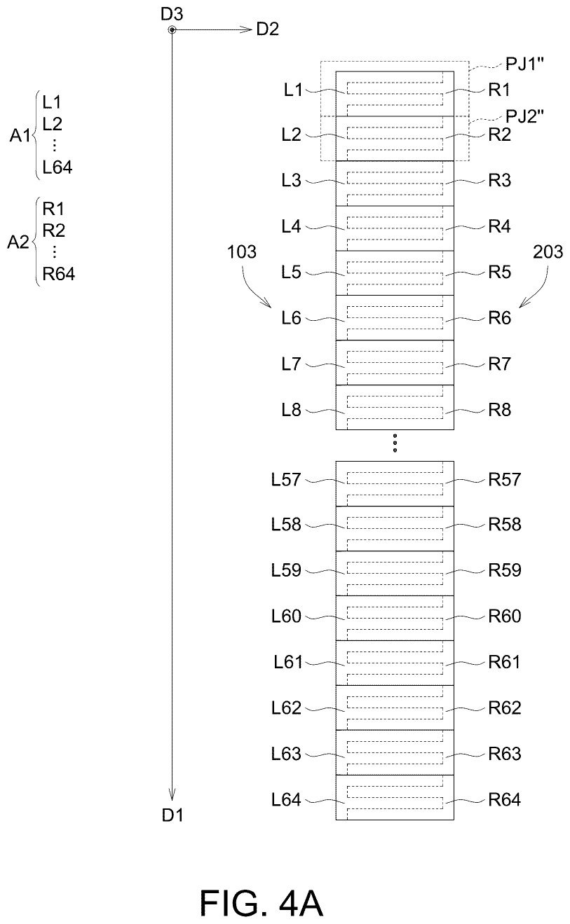

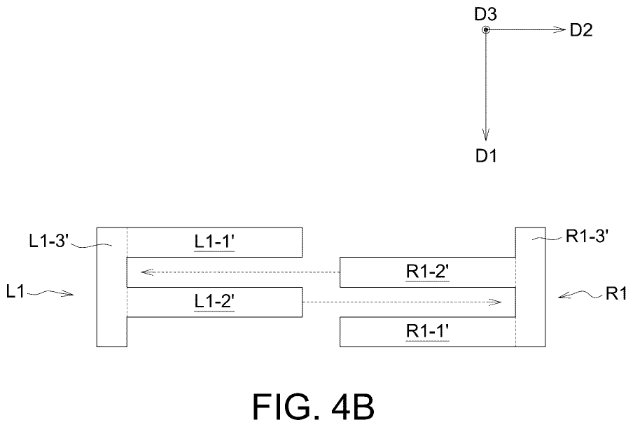

FIG. 4A is a top view of the transducer 103 and the transducer 203 according to another embodiment of the present disclosure. FIG. 4B is a schematic diagram showing projections of one of the array units L1-L64 and one of the array units R1-R64 in FIG. 4A (the array unit L1 and the array unit R1 are taken as examples for FIG. 4B). Please refer to both FIGS. 4A and 4B, each of the array units L1-L64 and the array units R1-R64 has a fork-shaped projection along the direction D3. Furthermore, the shape and area of the fork-shaped projection of each of the array units L1-L64 may be equal to the fork-shaped projection of each of the array units R1-R64. For example, the fork-shaped projection of the array unit R1 may be flipped along the direction D1 and then flipped along the direction D2 to obtain the fork-shaped projection of the array unit L1.

According to the above-mentioned fork-shaped projections, the array A1 and the array A2 are arranged in an interleaved manner (similar to a manner of fork-staggered), and one of the array units L1-L64 and the corresponding one of the array units R1-R64 may form a long strip projection. For example, the fork-shaped projection of array unit L1 and the corresponding fork-shaped projection of array unit R1 may form a long strip projection PJ1″, the fork-shaped projection of array unit L2 and the corresponding fork-shaped projection of array unit R2 may form a long strip projection PJ2″, and so on.

More specifically, the fork-shaped projection of the array unit R1 includes a first portion R1-1′, a second portion R1-2′ and a third portion R1-3′. The first portion R1-1′ and the second portion R1-2′ extend along the direction D2, and the third portion R1-3′ extends along the direction D1. The first portion R1-1′ is separated from the second portion R1-2′, and the third portion R1-3′ is connected to the first portion R1-1′ and the second portion R1-2′.

Similarly, the fork-shaped projection of the array unit L1 corresponding to the array unit R1 also includes a first portion L1-1′, a second portion L1-2′ and a third portion L1-3′. The first portion L1-1′ and the second portion L1-2′ extend along the direction D2, and the third portion L1-3′ extends along the direction D1. The first portion L1-1′ is separated from the second portion L1-2′, and the third portion L1-3′ is connected to the first portion L1-1′ and the second portion L1-2′.

The second portion R1-2′ with the fork-shaped projection of the array unit R1 is sandwiched between the first portion L1-1′ and the second portion L1-2′ with the fork-shaped projections of the corresponding array unit L1. Similarly, the second portion L1-2′ with the fork-shaped projection of the array unit L1 is sandwiched between the first portion R1-1′ and the second portion R1-2′ with the fork-shaped projections of the corresponding array unit R1. The step-shaped projection of the array unit L1 and the fork-shaped projection of the array unit R1 are separated from each other and form a long strip projection PJ1″.

The two sets of transducers in the above embodiments (e.g., the transducers 101 and 201 in FIG. 2A, the transducers 102 and 202 in FIG. 3A, and the transducers 103 and 203 in FIG. 4A) can simulate the functions of the linear array(s) and the phased array(s).

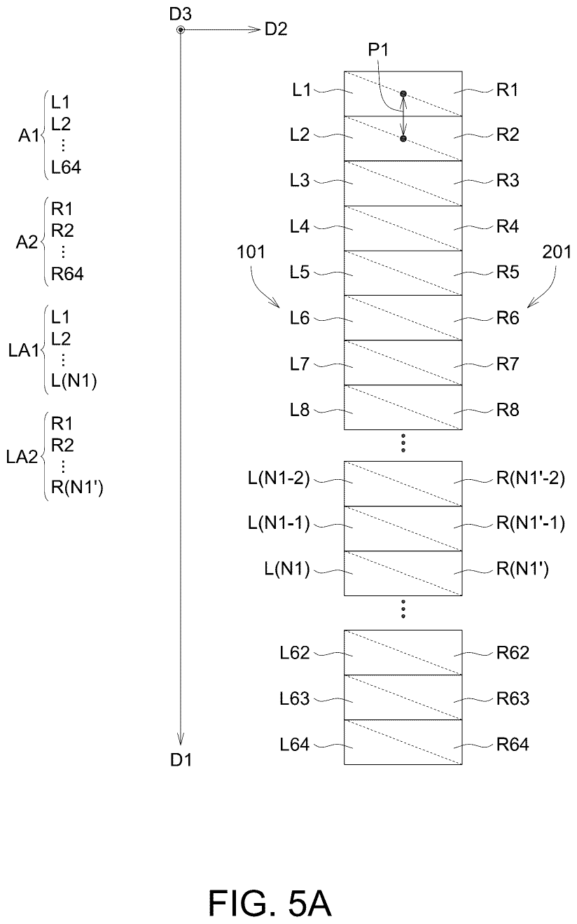

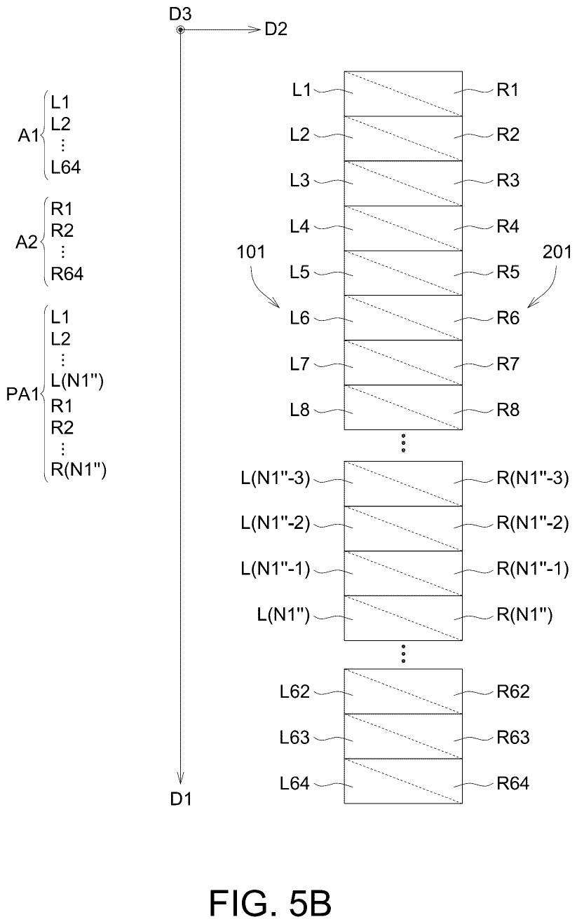

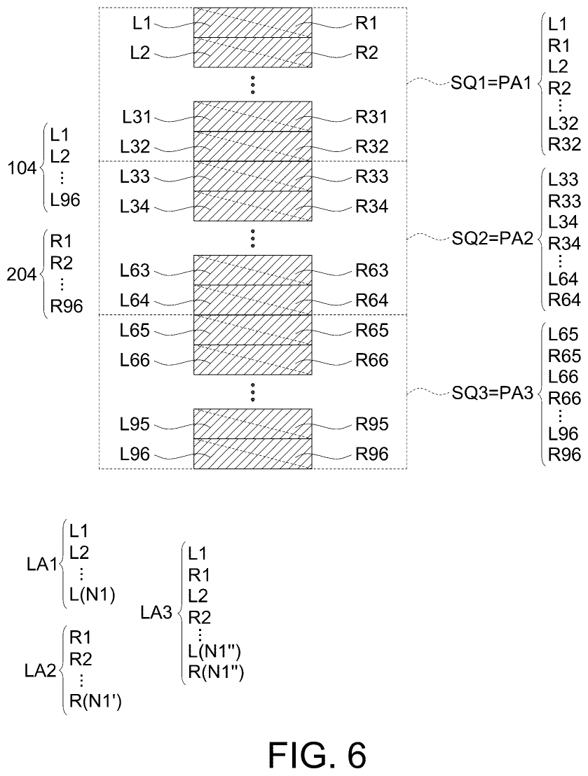

Please refer to FIG. 5A, which is a schematic diagram showing the transducers 101 and 201 in FIG. 2A simulate the functions of linear array(s) and phased array(s). In operation, only the transducer disposed on one side of the entire ultrasonic transducer 1000 may be driven, while the transducer disposed on the other side is not driven. For example, all or part of the array units L1-L64 of the transducer 101 on the left side are selectively driven to simulate one linear array. On the other hand, the array units R1-R64 on the right side are not driven. In the example of FIG. 5A, a first number N1 of array units L1-L(N1) of the transducer 101 on the left side are driven, so as to form a linear array LA1. The equivalent pitch of the linear array LA1 with respect to the direction D1 is twice the pitch P1. Furthermore, the linear array LA1 operates according to the center-line CL of FIG. 1B. Alternatively, a second number N1′ of array units R1-R(N1′) of the transducer 201 on the right side are driven, so as to form another linear array LA2. The equivalent pitch of the linear array LA2 according to the direction D1 is twice the pitch P1, and the linear array LA1 operates according to the center-line CL. That is, the linear array LA1 and the linear array LA2 of the present embodiment operate based on the same center-line CL and the same reference coordinate. Therefore, when the linear array LA1 and the linear array LA2 are switched with each other, or perform intersection/union operations (i.e., the images detected by the linear array LA1 and/or the linear array LA2 are processed by intersection/union image processing), there is no need to re-calibrate the center-line CL and the reference coordinate. The ultrasonic transducer 1000 with a single ultrasonic probe 100 can simulate at least two linear arrays, without the need to dispose more than two probes (e.g., dual probes). Furthermore, the length of the short axis Z of the multi-purpose ultrasound probe 100 can be reduced, so as to adapt to a scenario where a smaller part is to be detected (e.g., the intercostal part of the human body) and to facilitate operation of the user.

Next, please refer to FIG. 5B, which is a schematic diagram showing the transducer 101 and the transducer 201 in FIG. 2A simulate the function of phased array(s). In this embodiment, the transducer 101 on the left side and the transducer 201 on the right side are driven simultaneously, so as to simulate one phased array. For example, a third number N1″ of array units L1-L(N1″) in the transducer 101 on the left side are driven. Simultaneously, a third number N1″ of array units R1-R(N1″) in the transducer 201 on the right side are also driven. The array units L1-L(N1″) and array units R1-R(N1″) on both sides, which are driven simultaneously, may form a phased array PA1. The equivalent pitch of the phased array PA1 with respect to the direction D1 is equal to the pitch P1. Therefore, compared with the linear arrays LA1 and LA2 in FIG. 5A, the phased array PA1 in FIG. 5B has a smaller equivalent pitch. Furthermore, the phased array PA1 of FIG. 5B also operates based on the same center-line CL and reference coordinate as the linear array LA1 and the linear array LA2 of FIG. 5A. The ultrasonic transducer 1000 with a single ultrasonic probe 100 can perform the functions of the linear arrays LA1 and LA2 and the phased array PA1.

FIG. 6 is a schematic diagram showing the operations of the transducer 104 and the transducer 204 according to yet another embodiment of the present disclosure. The transducer 104 and the transducer 204 of the present embodiment are similar to the transducer 101 and the transducer 201 of FIG. 2A, except that the transducer 104 and the transducer 204 of the present embodiment include a great number of array units, for example, the transducer 104 includes 96 array units L1-L96, and the transducer 204 includes 96 array units R1-R96. The array units L1-L96 and the array units R1-R96 correspond to 192 channels, and the array units L1-L96 and the array units R1-R96 are switched in real time via the 192 channels, so as to transmit and/or receive ultrasonic signals.

Among the array units L1-L96 on the left side, all 96 array units may be driven, or a first number N1 of array units L1-L(N1) may be selectively driven, so as to form a linear array LA1. Similarly, all array units R1-R96 on the right side may be driven, or a second number N1′ of array units R1-R(N1′) may be selectively driven, so as to form another linear array LA2. The equivalent pitch of the single-sided driven linear array LA1 or LA2 is twice the pitch between adjacent two of array units L1-L96 (or between adjacent two of array units R1-R96). For example, the equivalent pitch of the linear array LA1 or the linear array LA2 is 0.4 mm, which is equal to one time of a water wavelength, which corresponds to an operating frequency of 3.75 MHz, as shown in equation (1).

$$0.4 mm = \{ ( 1.5 mm / μ s ) / 3.75 MHz \} \qquad ( 1 )$$

The equivalent pitch (i.e., 0.4 mm) of the linear array LA1 or the linear array LA2 is larger, and hence when the linear array LA1 or the linear array LA2 is used to simulate the function of a multi-focal lens, the transducer 104 or the transducer 204 has a larger lens aperture (for example, 38.4 mm) along the direction D1 (i.e., the direction of the short axis Z). Therefore, the detection result of the linear array LA1 or the linear array LA2 (i.e., the image obtained from the detection of the target object performed by the linear array LA1 or the linear array LA2) has a higher resolution.

The linear array LA1 and/or linear array LA2, which is single-sided driven, can perform intersection or union image operations, so as to eliminate blind spots when detecting target objects. For example, the linear array LA1 on one side is firstly driven to operate solely, detecting the target object to obtain a detection result. Then, the linear array LA1 and the linear array LA2 are double-sided driven to co-operate, detecting the target object to obtain a detection result. Then, the sole detection result by the linear array LA1 is synthesized with the co-operated detection result by the linear arrays LA1 and LA2, which can suppress or eliminate artifacts. The above operations are referred to as an operating mode of “linear array+linear array” (also referred to as a “LA+LA” operation mode).

Alternatively, all or part of the array units L1-L96 on the left side may co-operate with all or part of the array units R1-R96 on the right side, so as to form one linear array. For example, a third number N1 of array units L1-L(N1″) on the left side may co-operate with a third number N1 of array units R1-R(N1″) on the right side, so as to form a linear array LA3. The equivalent pitch of the linear array LA3 is half of the equivalent pitch of the linear array LA1 (or the linear array LA2), and such a smaller equivalent pitch can suppress artifacts.

On the other hand, in the “phased array operation mode”, all or part of the array units L1-L96 on the left side may co-operate with all or part of the array units R1-R96 on the right side, so as to form one phased array. More specifically, the array units L1-L96 and the array units R1-R96 may be divided into three sequence groups SQ1, SQ2 and SQ3. The sequence group SQ1 includes array units L1, R1, L2, R2, . . . , L31, R31, L32 and R32, a total of 64 array units. The 64 array units of the sequence group SQ1 are controlled by a 64-channel multiplexer to perform transmission and/or reception. The array units L1-L32 and the array units R1-R32 of the sequence group SQ1 form a phased array PA1.

Similarly, the sequence group SQ2 includes 64 array units L33, R33, L34, R34, . . . , L63, R63, L64 and R64, whose transmission and/or reception are controlled by a 64-channel multiplexer. The array units L33-L64 and the array units R33-R64 of the sequence group SQ2 form another phased array PA2. The sequence group SQ3 includes 64 array units L65, R65, L66, R66, . . . , L95, R95, L96 and R96, and the transmission and/or reception thereof are controlled by a 64-channel multiplexer. Furthermore, the array units L65-196 and the array units R65-R96 of the sequence group SQ3 form still another phased array PA3.

The equivalent pitch of each of the phased arrays PA1, PA2 and PA3 is half of the equivalent pitch of the linear array LA1 (or the linear array LA2). The equivalent pitch of each of the phased arrays PA1, PA2 and PA3 is smaller, which will suppress artifacts better. In one example, the equivalent pitch of each of the phased arrays PA1, PA2 and PA3 is, for example, equal to 0.2 mm, which is equal to half the water wavelength and corresponds to an operating frequency of 7.5 MHz, as shown in equation (2).

$$0.2 mm = \{ ( 1.5 mm / μ s ) / 7.5 MHz \} \qquad ( 2 )$$

Phased arrays PA1, PA2 and PA3 operate at higher operating frequencies and are suitable for detecting infants or small animals, as well as for detecting the heart or abdominal organs. Furthermore, when the phased arrays PA1, PA2, and PA3 are used to simulate the function of the multi-focal lens, the lens aperture of the transducer 101 or the transducer 201 along the direction D1 is, for example, 28.8 mm.

In the operation mode of “phased array+phased array” (also referred to as the operation mode of “PA+PA”), the three phased arrays PA1, PA2 and PA3 may perform a union operation. For example, respective images obtained from respective detections of the target object by the three phased arrays PA1, PA2 and PA3 may be synthesized with three-beam, which can more effectively suppress or eliminate the artifacts.

Alternatively, the system may operate in an operation mode of “linear array+phased array” (i.e., an operating mode of “LA+PA”). The detection results of the single-sided driven linear array LA1 or linear array LA2 can be synthesized with the detection results of the phased arrays PA1, PA2 or PA3, and the artifacts can also be eliminated.

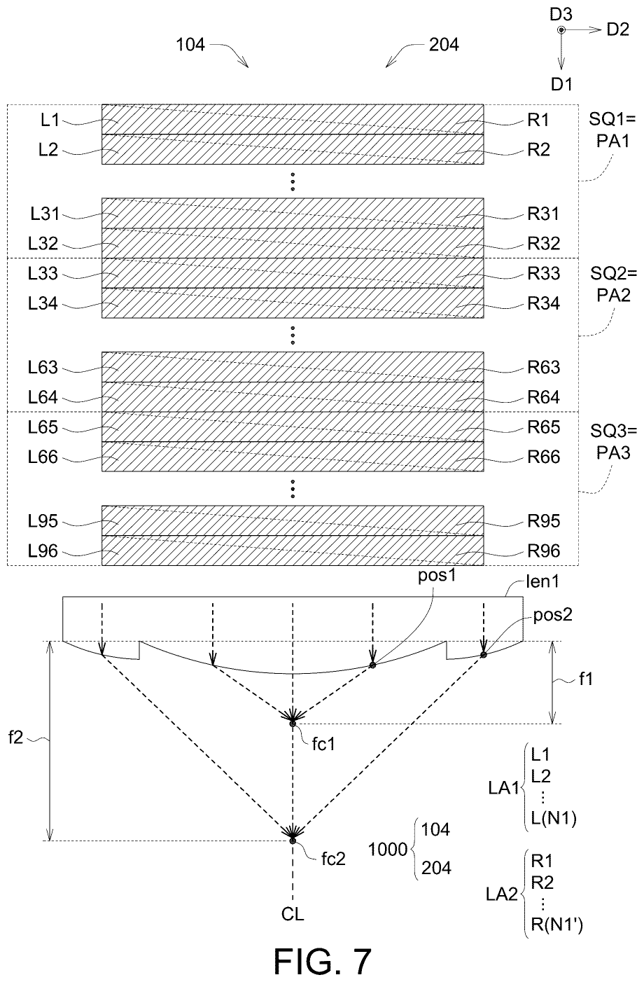

Next, please refer to FIG. 7, which is a schematic diagram showing the transducer 104 and the transducer 204 in FIG. 6 are simulated as a multi-focal lens len1. The multi-focal lens len1 is, for example, a “Fresnel lens”. The multi-focal lens len1 has different focal lengths at different reference positions, which can focus the ultrasonic signals generated by the phased arrays PA1, PA2 and PA3 and the linear arrays LA1 and LA2 at multiple focal points of different focal lengths.

The multi-focal lens len1 has a first reference position pos1 and a second reference position pos2 along the direction D2. The linear arrays LA1 and/or LA2 may operate according to the first reference position pos1, while the phased arrays PA1, PA2 and/or PA3 may operate according to the second reference position pos2. The first reference position pos1 is closer to the center-line CL of the transducer 104 and the transducer 204. The second reference position pos2 is farther away from the center-line CL. The second reference position pos2 corresponds to one of the two sides of the transducers 104 and 204.

The first reference position pos1 corresponds to a first focal length f1 of the multi-focal lens len1, and is capable of focusing the ultrasonic signals of the linear arrays LA1 and/or LA2 at a focal point fc1 with the first focal length f1. The first focal length f1 is, for example, 20 mm. The second reference position pos2 corresponds to a second focal length f2 of the multi-focal lens len1, and is capable of focusing the ultrasonic signals of the phased arrays PA1, PA2 and/or PA3 at a focal point fc2 with the second focal length f2. The second focal length f2 is, for example, 64 mm. In this embodiment, the first focal length f1 is smaller than the second focal length f2.

In operation, since the width of the multi-focal lens len1 increases, the side lobe of the artifact becomes larger. By adjusting the curvatures of both sides of the multi-focal lens len1 and the first focal length f1 of the near field, the artifacts formed by the energy of the ultrasonic signals on both sides can be deflected to a far location, thereby improving the artifacts in the near field. Furthermore, in the far field, the energy of the ultrasonic signals can be transmitted to the far location, so as to improve the penetration depth of the ultrasonic signals.

On the other hand, the second focal length f2 of the phased arrays PA1, PA2 and PA3 and the operating frequency of the phased arrays PA1, PA2 and PA3 can be dynamically adjusted, so as to simulate a convex linear array (CLA) which can improve the resolution of the images detected by the phased arrays PA1, PA2 and PA3 and hence obtain an image similar to that obtained by a convex linear array. For example, the operating frequencies of the phased arrays PA1, PA2 and PA3 are adjusted to 3.75 MHz to simulate the operating frequency of the convex linear array. Furthermore, the simulated convex linear array is reduced from a commonly used convex angle of 73 degrees to a convex angle less than 60 degrees.

More specifically, a dynamic focusing mechanism may be implemented on the phased arrays PA1, PA2 and PA3 based on the beamforming technology, and focal positions of the phased arrays PA1, PA2 and PA3 may be adjusted at different depths in the direction D1, so as to simulate an imaging effect of the convex linear array. Alternatively, a dynamic aperture mechanism may be implemented on the phased arrays PA1, PA2 and PA3 according to the beamforming technology, and the number of array units is adjusted at different depths in the direction D1, so as to maintain a recognition-rate and a signal-to-noise ratio (SNR) of the image.

In another example, an image reconstruction algorithm can be implemented based on digital signal processing technology and performed on the images detected by the phased arrays PA1, PA2 and PA3, so as to re-construct an image similar to that obtained by a convex linear array. Alternatively, when receiving the ultrasonic signals of the phased arrays PA1, PA2 and PA3, a mechanism of delaying and weighting may be implemented to simulate an echo collection method of the convex linear array.

In another example, hardware settings of the array units L1-L96 and the array units R1-R96 can be adjusted, and geometric arrangements of the probes of the phased arrays PA1, PA2 and PA3 can be adjusted in physical positions, so as to simulate arrangement characteristics of the convex linear array. Alternatively, a soft simulation may be performed according to post-processing technology for the image, so as to re-arrange and adjust the images detected by the phased arrays PA1, PA2 and PA3, thereby simulating the imaging effect achieved by the convex linear array.

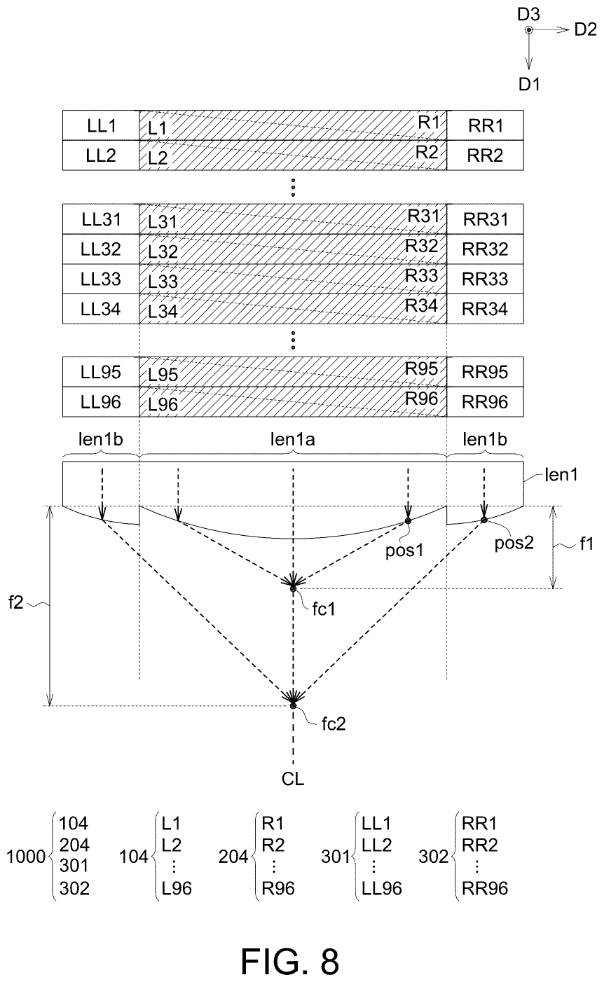

Next, please refer to FIG. 8, which is a schematic diagram showing another embodiment in which the ultrasonic transducer 1000 is simulated as the multi-focal lens len1. The ultrasonic transducer 1000 of this embodiment is similar to the ultrasonic transducer 1000 of FIG. 7, except that the ultrasonic transducer 1000 of this embodiment further includes a transducer 301 and a transducer 302, in addition to the transducers 104 and 204. The transducer 301 and transducer 302 may be referred to as “third transducers”. The transducer 301 and the transducer 302 are disposed at the periphery of the transducer 104 and the transducer 204 along the direction D2.

The transducer 301 includes the array units LL1-LL96, and the transducer 302 includes the array units RR1-RR96. Different from the triangular projections of the array units L1-L96 of the transducer 104 and the array units R1-R96 of the transducer 204, each of the array units LL1-LL96 and the array units RR1-RR96 has a long strip projection along the direction D3. The array units LL1-LL96 of the transducer 301 are correspondingly arranged on one side of the array units L1-L96 of the transducer 104. For example, the array unit LL1 is arranged adjacent to one side of the array unit L1, and the array unit LL2 is arranged adjacent to one side of the array unit L2, and so on. Similarly, the array units RR1-RR96 of the transducer 302 are correspondingly arranged on one side of the array units R1-R96 of the transducer 204.

In operation, the transducer 104 and the transducer 204 can be simulated as linear array(s). At the same time, the transducer 301 and the transducer 302 disposed at the periphery may be simulated as phased array(s). Furthermore, the multi-focal lens len1 includes a first focal portion len1a and a second focal portion len1b. The first focal portion len1a corresponds to the transducer 104 and the transducer 204 along the direction D1. The second focal portion len1b corresponds to the transducer 301 and the transducer 302 along the direction D1.

It will be apparent to those skilled in the art that various modifications and variations can be made to the disclosed embodiments. It is intended that the specification and examples be considered as exemplars only, with a true scope of the disclosure being indicated by the following claims and their equivalents.

What is claimed is:

1. An ultrasonic transducer, comprising:
a first transducer, comprising:
a plurality of first array units, arranged along a first direction to form a first array, wherein the first direction is a direction of a long axis of the ultrasonic transducer; and
a second transducer comprising:
a plurality of second array units, arranged along the first direction to form a second array, the second array and the first array are disposed in an interleaving manner and separated from each other, wherein one of the second array units is sandwiched between corresponding adjacent two of the first array units,
wherein the first transducer and the second transducer have a common reference coordinate, and the first transducer and the second transducer have a function of a multi-focal lens along a second direction, the second direction is a direction of a short axis of the ultrasonic transducer and is substantially perpendicular to the first direction.

2. The ultrasonic transducer of claim 1, wherein each of the first array units and each of the second array units has a projection along a third direction, the third direction is substantially perpendicular to the first direction and the second direction, and the projection has a triangular shape, a step shape or a fork shape.

3. The ultrasonic transducer of claim 2, wherein the projection with the triangular shape has a short side along the first direction, a long side along the second direction, and a hypotenuse connected to the short side and the long side.

4. The ultrasonic transducer of claim 3, wherein the hypotenuse of the projection of one of the first array units is adjacent to the hypotenuse of the projection of a corresponding one of the second array units.

5. The ultrasonic transducer of claim 4, wherein the projection of the first array unit and the projection of the corresponding one of the second arrays unit form a long strip projection.

6. The ultrasonic transducer of claim 2, wherein the projection with the step shape comprising:
a first portion, extending along the second direction, and having a first width along the first direction; and
a second portion, extending along the second direction, and having a second width along the first direction, wherein the second width is greater than the first width.

7. The ultrasonic transducer of claim 6, wherein the first portion and the second portion of the projection of one of the first array units are respectively adjacent to the second portion and the first portion of the projection of the corresponding one of the second array units, and the projection of the first array unit and the projection of the corresponding one of the second array units form a long strip projection.

8. The ultrasonic transducer of claim 2, wherein the projection with the fork shape comprising:
a first portion, extending along the second direction;
a second portion, extending along the second direction; and
a third portion, extending in the first direction,
wherein the first portion is separated from the second portion, and the third portion is connected to the first portion and the second portion.

9. The ultrasonic transducer of claim 8, wherein the second portion of the projection of one of the first array units is sandwiched between the first portion and the second portion of the projection of the corresponding one of the second array units, and the projection of the first array unit and the projection of the corresponding one of the second array units form a long strip projection.

10. The ultrasonic transducer of claim 1, wherein the multi-focal lens is a Fresnel lens, the multi-focal lens has a first reference position and a second reference position along the second direction, the first reference position corresponds to a first focal length, the second reference position corresponds to a second focal length, and the first focal length is smaller than the second focal length.

11. The ultrasonic transducer of claim 10, wherein the first reference position of the multi-focal lens corresponds to a center-line of the ultrasonic transducer, and the second reference position corresponds to one of two sides of the ultrasonic transducer.

12. The ultrasonic transducer of claim 10, wherein the first transducer and the second transducer are selectively driven to form a linear array and/or a phased array, the linear array has the first focal length, and the phased array has the second focal length.

13. The ultrasonic transducer of claim 12, wherein a first number of the first array units of the first transducer or a second number of the second array units of the second transducer are driven to form the linear array.

14. The ultrasonic transducer of claim 13, wherein there is a first pitch between adjacent two of the first array units or between adjacent two of the second array units, and the linear array corresponds to a first equivalent pitch which is equal to twice the first pitch.

15. The ultrasonic transducer of claim 12, wherein a third number of the first array units of the first transducer and the third number of the second array units of the second transducer are driven to form the phased array.

16. The ultrasonic transducer of claim 15, wherein there is a first pitch between adjacent two of the first array units or between adjacent two of the second array units, and the phased array corresponds to a second equivalent pitch which is equal to the first pitch.

17. The ultrasonic transducer of claim 15, wherein the phased array has a first operating frequency, and the phased array dynamically adjusts the second focal length and the first operating frequency to simulate a convex linear array.

18. The ultrasonic transducer of claim 1, further comprises a plurality of third transducers, wherein the third transducers are disposed at a periphery of the first transducer and the second transducer along the second direction, wherein the first transducer and the second transducer form a linear array, and the third transducers form a phased array.

19. The ultrasonic transducer of claim 18, wherein the multi-focal lens comprises a first focal portion and a second focal portion, wherein the first focal portion corresponds to the first transducer and the second transducer along the first direction, and the second focal portion corresponds to the third transducers along the first direction.

20. An ultrasonic probe, comprising:
a gripping handle; and
an ultrasonic transducer of claim 1,
wherein the ultrasonic transducer is disposed at one end of the gripping handle.
